(19) United States
(12) Patent Application Publication
(10) Pub. No.: US 2021/0077714 A1
(43) Pub. Date: Mar. 18, 2021

(54) IMPLANTABLE SYSTEM FOR INCREASING INTRATHECAL DRUG DISPERSION

(71) Applicant: Medtronic, Inc., Minneapolis, MN (US)

(72) Inventor: Jeffrey Bodner, Plymouth, MN (US)

(21) Appl. No.: 16/572,763

(22) Filed: Sep. 17, 2019

(51) Int. Cl. A61M 5/142; A61M 5/172; A61M 39/02

(52) U.S. Cl. CPC A61M 5/14276; A61M 5/1723; A61M 39/0208; A61M 2205/0294; A61M 2230/06; A61M 2210/1003; A61M 2039/0244; A61M 2205/8243; A61M 2205/3334; A61M 2202/0464; A61M 2210/0693; A61M 2230/42

(57) ABSTRACT

A medical device configured to improve medicament dispersion within a cerebrospinal fluid of a patient. The medical device including an implantable catheter having a distal end configured to be positioned within a flow of the cerebrospinal fluid, a proximal end, a body defining a lumen extending lengthwise along the implantable catheter configured to enable a flow of medicament from the proximal end to an infusion port located in proximity to the distal end, and a piezoelectric element positioned in proximity to the infusion port configured to selectively oscillate during medicament administration to improve dispersion of the medicament within the cerebrospinal fluid.

### FIELD

The present technology is generally related to implantable medical devices, and more particularly to a system and method utilizing an implantable catheter having a piezoelectric tip for increased drug dispersion into the cerebrospinal fluid of a patient.

### BACKGROUND

Implantable medical devices, such as an implantable access port or medical pump, are useful in managing the delivery and dispensation of prescribed therapeutic agents, nutrients, drugs, medicaments such as antibiotics, blood clotting agents, analgesics and other fluid and/or fluid like substances (collectively “medicaments” or “infusates”) to patients in volume- and time-controlled doses. Such implantable devices are particularly useful for treating diseases and disorders that require regular or chronic (i.e., long-term) pharmacological intervention, including tremor, spasticity, multiple sclerosis, Alzheimer's disease, Parkinson's disease, amyotrophic lateral sclerosis (ALS), Huntington's disease, cancer, epilepsy, chronic pain, urinary or fecal incontinence, sexual dysfunction, obesity, and gastroparesis, to name just a few. Depending upon their specific designs and intended uses, implantable devices are well adapted to administer infusates to specific areas within the central nervous system, including the subarachnoid, epidural, intrathecal, and intracranial spaces.

Administration of infusates directly into the cerebrospinal fluid of a patient has a number of important advantages over other forms of medicament administration. For example, oral administration is often not workable because the systematic dose of the substance needed to achieve the therapeutic dose at the target site may be too large for the patient to tolerate without adverse side effects. Also, some substances simply cannot be absorbed in the gut adequately for a therapeutic dose to reach the target site. Moreover, substances that are not lipid soluble may not cross the blood-brain barrier adequately if needed in the brain. In addition, infusion of substances from outside the body requires a transcutaneous catheter, which results in other risks such as infection or catheter dislodgment.

Typically, such implantable medical devices include an implantable catheter in fluid communication with an implantable access port or an implantable pump. Implantable access ports are typically placed cranially or over the ribs, and are connected to a catheter which is surgically placed in the intraventricular space of the brain or intraspinal area of the spinal cord. When it is desirable to administer a medicament, a needle is inserted through the patient's skin, through a septum of the port, which is in fluid connection with the catheter. The medicament is then injected into the port where it passes through the catheter and into the patient's cerebrospinal fluid.

Implantable pumps are typically implanted at a location within the body of a patient (typically a subcutaneous region in the lower abdomen), and are connected to a catheter configured to deliver medicament to a selected delivery site in the patient. Such implantable medical pumps typically include an expandable fluid reservoir, which is accessible for refill etc. through an access port. Medicament flows from the reservoir through the catheter and into the patient's cerebrospinal fluid according to programmed parameters.

The catheter is generally configured as a flexible tube with a lumen running the length of the catheter to a selected delivery site in the body, such as the subarachnoid space. Drug molecules exiting the catheter lumen flow into the subarachnoid space, and begin mixing with the cerebrospinal fluid. Frequently, the drug exits the catheter slowly (e.g., at a flow rate of 1 mL per hour or less), where it tends to stagnate in the slow-moving cerebrospinal fluid immediately surrounding the catheter. This slow-moving fluid is known to those schooled in the science of fluid mechanics as a boundary-layer, which is a consequence of friction between a viscous fluid and a surface (i.e. the catheter). A slow or delayed mixing of the drug with the cerebrospinal fluid can decrease the efficacy of the drug and resultant therapeutic effect. Although various attempts have been made to improve medicament dispersion within the cerebrospinal fluid, it is desirous to further improve the efficiency of drug delivery into the cerebrospinal fluid of the patient. Applicants of the present disclosure have developed a system and method to address this concern.

### SUMMARY

The techniques of this disclosure generally relate to implantable systems and methods configured to improve medicament dispersion within a flow of cerebrospinal fluid of a patient through the use of an implantable catheter having a piezoelectric element configured to selectively oscillate during medicament administration to impart a fluid motion in the cerebrospinal fluid and medicament surrounding the implantable catheter to encourage dispersion of the medicament surrounding a slower moving flow of cerebrospinal fluid within a boundary layer immediately surrounding the implantable catheter. Accordingly, embodiments of the present disclosure optimize current therapy techniques by encouraging a more rapid dispersion through an active mixing of the medicament with the cerebrospinal fluid. Although applications of the present disclosure can be used for the delivery of any type of medicament, it is believed that the present disclosure may be particularly useful in targeting specific proteins or viruses as a root cause of a particular disease or disorder, as opposed to merely addressing undesirable symptoms.

One embodiment of the present disclosure provides a medical device configured to improve medicament dispersion. The medical device can include an implantable catheter having a distal end configured to be positioned within a flow of cerebrospinal fluid of the patient, a proximal end, and a body defining a lumen extending lengthwise along the implantable catheter configured to enable a flow of medicament from the proximal end to an infusion port position in proximity to the distal end, the implantable catheter further including a piezoelectric element positioned in proximity to the infusion port configured to selectively oscillate during medicament administration to improve dispersion of the medicament within the cerebrospinal fluid.

In one embodiment, the piezoelectric element can include an oscillating surface configured to impart fluid motion to the cerebrospinal fluid and medicament surrounding the implantable catheter during administration of the medicament. In one embodiment, the piezoelectric element is configured to encourage dispersion of the medicament beyond a slower moving flow of cerebrospinal fluid within a boundary layer immediately surrounding the implantable catheter. In one embodiment, the piezoelectric element is configured to oscillate for a predefined period of time in relation to administration of the medicament. In one embodiment, the predefined period of time is in a range of between about 15 seconds and about 30 seconds.

In one embodiment, the medical device further includes one or more physiological sensor configured to monitor one or more physiological conditions of the patient to time oscillation of the piezoelectric element to correspond with an inference of heightened cerebrospinal fluid oscillations. In one embodiment, the one or more physiological sensor is configured to monitor at least one of a heart rate or respiratory rate of the patient.

In one embodiment, the proximal end of the implantable catheter is operably coupled to an implantable port configured to subcutaneously receive medicament. In one embodiment, the medical device further comprises a needle detection sensor configured to detect an insertion of a needle into the implantable port to time oscillation of the piezoelectric element. In one embodiment, the proximal end of the implantable catheter is operably coupled to an implantable pump having a medicament reservoir. In one embodiment, the medical device further comprises a medicament flow sensor configured to detect a flow of medicament to time oscillation of the piezoelectric element.

In one embodiment, the medical device further includes an implantable power source configured to power the piezoelectric element. In one embodiment, the implantable power source is configured to be inductively charged through a skin of the patient. In one embodiment, the implantable power source is positioned in proximity to a proximal end of the implantable catheter. In one embodiment, the implantable catheter includes one or more electrical conduit electrically coupling the implantable power source to the piezoelectric element. In one embodiment, the body of the implantable catheter defines one or more electrical conduit lumen extending lengthwise along the implantable catheter configured to house the one or more electrical conduit.

Another embodiment of the present disclosure provides a medical device configured to improve medicament dispersion within a flow of cerebrospinal fluid of the patient. The medical device can include an implantable catheter and port. The implantable catheter can have a distal end configured to be positioned within the flow of cerebrospinal fluid, a proximal end, and a body defining a lumen configured to enable a flow of medicament into an infusion port positioned in proximity to the distal end, and a piezoelectric element positioned in proximity to the infusion port. The implantable port can be in fluid communication with the implantable catheter and can be configured to receive medicament from a medicament source. The piezoelectric element can include an oscillating surface configured to impart fluid motion in the cerebrospinal fluid and medicament surrounding the implantable catheter to encourage dispersion of the medicament beyond a slower moving flow of cerebrospinal fluid within a boundary layer immediately surrounding the implantable catheter.

Another embodiment of the present disclosure provides a method of improving medicament dispersion, including: administering medicament into a flow of cerebrospinal fluid of a patient via an implantable catheter having a distal end configured to be positioned within the flow of cerebrospinal fluid, a proximal end, a body defining a lumen configured to enable a flow of medicament to an infusion port positioned in proximity to the distal end, and a piezoelectric element positioned in proximity to the infusion port; and selectively oscillating a surface of the piezoelectric element to impart fluid motion in the cerebrospinal fluid and medicament surrounding the implantable catheter to encourage dispersion of the medicament beyond a slower moving flow of cerebrospinal fluid within a boundary layer immediately surrounding the implantable catheter.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

The details of one or more aspects of the disclosure are set forth in the accompanying drawings and the description below. Other features, objects, and advantages of the techniques described in this disclosure will be apparent from the description in the drawings, and from the claims.

### BRIEF DESCRIPTION OF THE DRAWINGS

The disclosure can be more completely understood in consideration of the following detailed description of various embodiments of the disclosure, in connection with the accompanying drawings, in which:

FIG. 1A depicts a medical device configured to improve medicament dispersion having a catheter inserted into an intracranial space of a patient's brain, in accordance with an embodiment of the disclosure.

FIG. 1B depicts a medical device configured to improve medicament dispersion having a catheter inserted into an intrathecal space of a patient's spinal column, in accordance with an embodiment of the disclosure.

FIG. 2 is a perspective view depicting a medical device including a catheter and an implantable port, in accordance with an embodiment of the disclosure.

FIG. 3 is a partial, cross-sectional view depicting the medical device of FIG. 2.

FIG. 4 depicts a partial cross-sectional view of the catheter of the medical device of FIG. 3.

FIG. 5A is a perspective view depicting a catheter inserted into a subarachnoid space of a patient, in accordance with an embodiment of the disclosure.

FIG. 5B depicts the dispersion of medicament within the cerebrospinal fluid of a patient after approximately 7.5 seconds of initiating infusion from the catheter of FIG. 5A at a rate of 1 mL per hour.

FIG. 5C depicts the dispersion of medicament within the cerebrospinal fluid of a patient after approximately 15 seconds of initiating infusion from the catheter of FIG. 5A at a rate of 1 mL per hour.

FIG. 6A is a perspective view depicting a catheter with an activated piezoelectric element inserted into a subarachnoid space of a patient, in accordance with an embodiment of the disclosure.

FIG. 6B depicts the dispersion of medicament within the cerebrospinal fluid of a patient after approximately 7.5 seconds of initiating infusion from the catheter of FIG. 5A at a rate of 1 mL per hour.

FIG. 6C depicts the dispersion of medicament within the cerebrospinal fluid of a patient after approximately 15 seconds of initiating infusion from the catheter of FIG. 6A at a rate of 1 mL per hour.

FIG. 7 is a perspective, exploded view depicting a medical device including a catheter and an implantable pump, in accordance with an embodiment of the disclosure.

FIG. 8 depicts a block diagram of medical device of FIG. 7.

FIG. 9 depicts a medical device operably coupled to one or more external components, in accordance with an embodiment of the disclosure.

FIG. 10A is a partial, perspective view depicting a catheter having a bending plate, in accordance with an embodiment of the disclosure.

FIG. 10B is a partial, cross-sectional view depicting the catheter of FIG. 10A.

FIG. 10C is a perspective view depicting the catheter of FIG. 10A inserted into a subarachnoid space of a patient.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

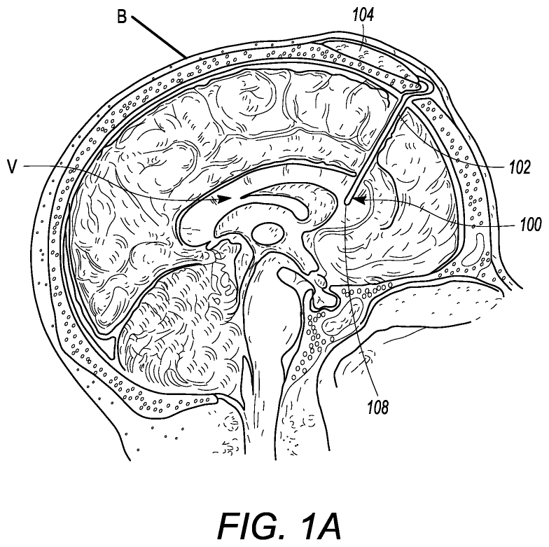

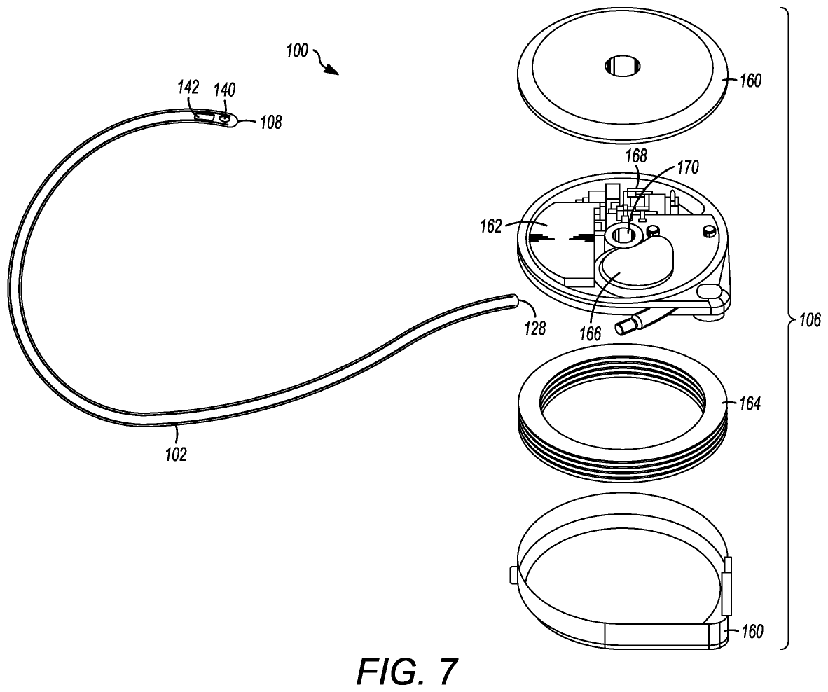

Referring to FIGS. 1A and 1B, a medical device 100 configured to improve medicament dispersion is depicted in accordance with an embodiment of the disclosure. The medical device 100 can include an implantable catheter 102, which in some embodiments, can be in fluid communication with either an implantable port 104 (as depicted in FIGS. 1A-B) or an implantable pump 106 (as depicted in FIG. 7). As depicted, the medical device 100 can be implanted within the body B of a patient. In some embodiments, a distal tip 108 of the implantable catheter 102 can optionally be surgically implanted in a ventricle V in the patient's brain (as depicted in FIG. 1A) or in a patient's intraspinal space (as depicted in FIG. 1B). The implantable port 104 or implantable pump 106 can be placed cranially (as depicted in FIG. 1A), or in an interior torso cavity or in proximity to the patient's ribs (as depicted in FIG. 1B). In either case, the implantable port 104 or implantable pump 106 is typically placed subcutaneously, and can be held in position by sutures or other retaining features.

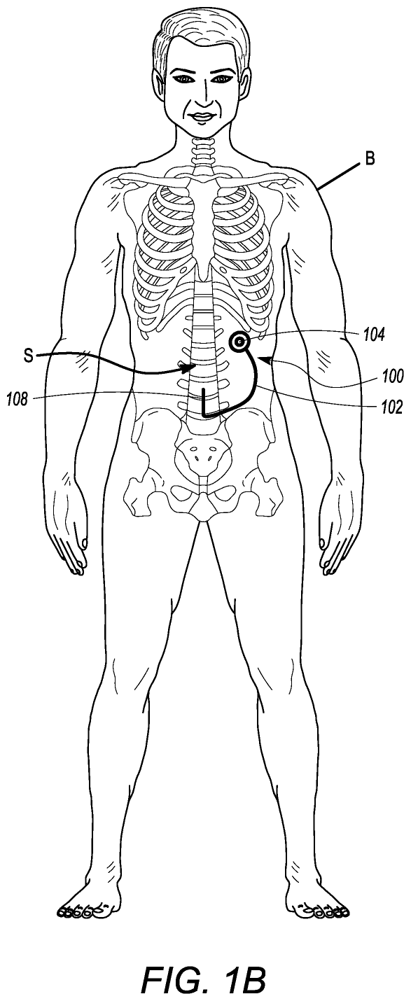

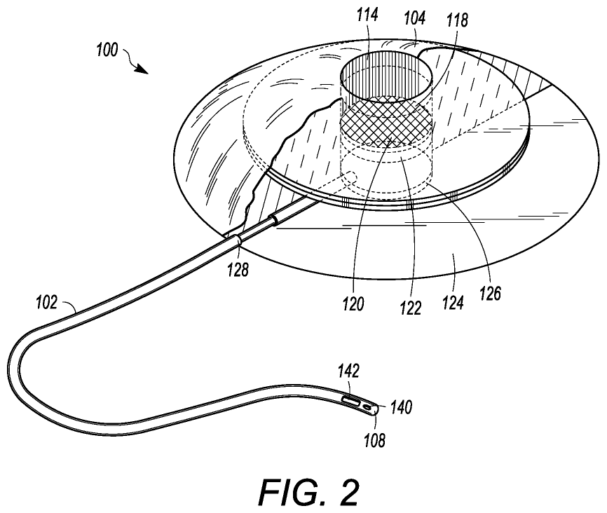

With additional reference to FIGS. 2 and 3, an embodiment of a medical device 100 including an implantable catheter 102 operably coupled to an implantable port 104 is depicted. In particular, FIG. 2 depicts a perspective view of the medical device 100, while FIG. 3 depicts a cross-sectional schematic view of the medical device 100. The implantable port 104 can include a generally dome-shaped upper housing 110 and a disk-shaped lower housing 112. Upper and lower housings 110, 112 can be constructed of a body-tolerant material such as titanium or a body-compatible plastic such as silicone rubber, and sealed to one another about their periphery.

The upper housing 110 can include a centrally-located first septum 114. The first septum 114 can define an upper boundary of a first chamber 116. A chamber wall 118, which in some embodiments is substantially cylindrical in shape, can define the walls of the first chamber 116. The chamber wall 118 can be made of a rigid material, such as a biocompatible polymer or titanium. A needle screen 120 can be positioned opposite of the first septum 114 to define a lower boundary of the first chamber 116. In some embodiments, the needle screen 120 can inhibit needles having a diameter larger than a given diameter from passing therethrough while allowing needles having diameters that are smaller than the given diameter to pass therethrough. In one embodiment, the needle screen 120 is a mesh screen constructed of wire configured to enable needles of a 25-gauge or smaller to pass, while inhibiting needles having a diameter larger than 25-gauge from passing therethrough.

A second septum 122 can be positioned immediately adjacent to and below the needle screen 120. The second septum 122 can define an upper boundary of a second chamber 124. In one embodiment, the first septum 114 and the second septum 122 can be constructed of a resilient, pliable material such as a self-sealing silicone rubber. The chamber wall 118 can define the walls of the second chamber 124. A needle stop 126 can be positioned opposite the second septum 122 to define a lower boundary of the second chamber 124. The needle stop 126 can be configured to inhibit a needle from passing entirely through the second chamber 124. In one embodiment, the needle stop 126 can be constructed of a rigid, biocompatible polymer material. In some embodiments, the needle stop 124 rests on the lower housing 112.

In one embodiment, the implantable catheter 102 can be connected to the implantable port 104 by sliding a proximal end 128 of the catheter 102 over a catheter connector 130 of the implantable port 104. The catheter connector 130 can be in fluid communication with the second chamber 124 via conduit 132. Accordingly, a quantity of medicament can pass from a syringe external to the patient, through the implantable port 104 to a distal end 108 of the catheter 102. In particular, to administer medicament, a needle of a syringe filled with the medicament can be passed through a patient's skin, the first septum 114, the needle screen 120, and the second septum 122 to enter into the second chamber 124. As the medicament is expelled from the syringe, the medicament fills the second chamber 124, passes through the conduit 132 and into a lumen 136 generally extending lengthwise within a body 138 of the catheter 102 between the proximal end 128 and an infusion port 140 in proximity to the distal end 108. In some embodiments, the infusion port 140 can be positioned on the distal end or tip 108 of the catheter 102. Alternatively, as depicted, the infusion port 140 can be positioned proximately from the distal tip 108 along the body 138 of the catheter 102.

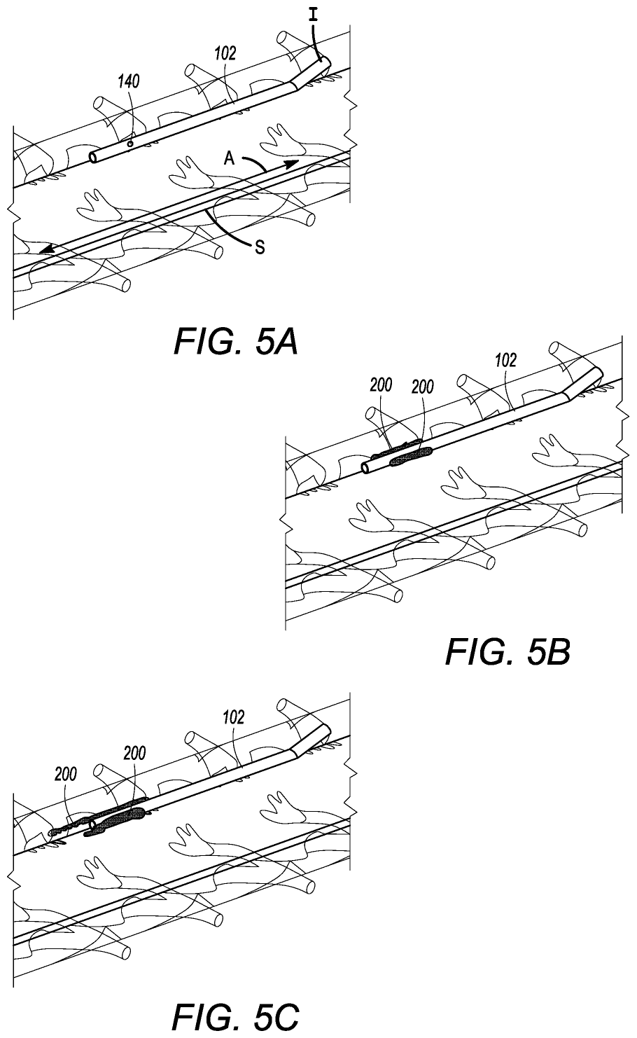

FIG. 5A depicts a catheter 102 positioned within a subarachnoid space of a patient. In particular, the catheter 102 enters the subarachnoid space at an insertion site I, and extends substantially parallel to a longitudinal axis A of the patient's spinal column S, thereby enabling intrathecal delivery of medicament through an infusion port 140 of the catheter 102. FIGS. 5B and 5C depict the catheter 102 as the medicament 200 exits the infusion port 140 and flows into the subarachnoid space. Specifically, FIG. 5B depicts the dispersion of medicament 200 after approximately 7.5 seconds of initiating infusion, and FIG. 5C depicts the dispersion of medicament 200 after approximately 15 seconds of initiating infusion.

As the medicament 200 exits the infusion port 140 and flows into the subarachnoid space, the medicament 200 begins mixing with the cerebrospinal fluid. Where the medicament 200 is expelled from the infusion port 140 at a relatively slow rate (e.g., a flow rate of 1 mL per hour), the medicament 200 commonly stagnates in the slow-moving cerebral spinal fluid immediately surrounding the catheter 102. Although the pulsatile flow of the cerebrospinal fluid eventually causes the medicament 200 to drift away from the catheter 102 into faster moving cerebrospinal fluid, proper mixing of the medicament 200 into the cerebrospinal fluid can take several minutes. A slow or delayed mixing of the medicament 200 with the cerebrospinal fluid can decrease the efficacy of the medicament 200, as well as the resultant therapeutic effect.

With continued reference to FIGS. 2 and 3, in order to encourage a quicker dispersion of the medicament 200 beyond the slower moving flow of cerebrospinal fluid within a boundary layer immediately surrounding the implantable catheter, embodiments of the present disclosure can include a piezoelectric element 142 positioned on the implantable catheter 102. In some embodiments, the piezoelectric element 142 can be positioned near the infusion port 140 in proximity to a distal end 108 of the catheter 102, and can include an oscillating surface 144 configured to impart fluid motion in the cerebrospinal fluid and medicament surrounding the implantable catheter 102 during administration of the medicament, through a phenomenon referred to as “steady streaming.”

In some embodiments, the piezoelectric element 142 can be powered by a power source 146, which can be incorporated into the implantable port 104 or other implantable device to which the implantable catheter 102 is operably coupled, such as an implantable pump 106 (as depicted in FIG. 7). In other embodiments, the power source 146 can be generally positioned subcutaneously, transcutaneously, epiccutaneously or supracutaneously in proximity to a proximal end of the implantable catheter 102. In some embodiments, the power source 146 is configured to be wirelessly charged through the skin of the patient via an induction coil 148.

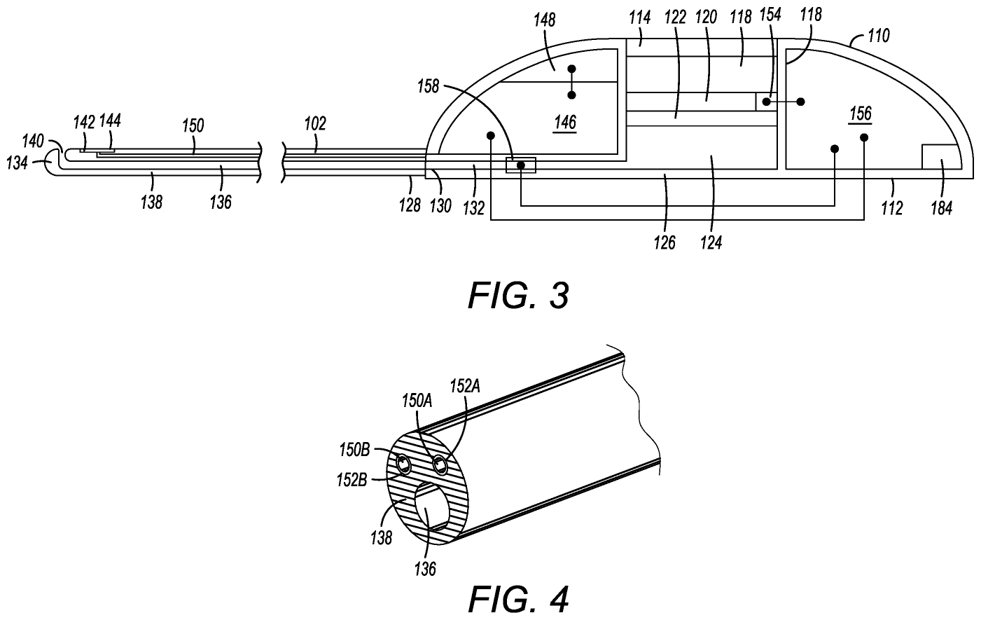

One or more electrical conduit 150 extending lengthwise along the implantable catheter parallel to the lumen 136 can electrically couple the piezoelectric element 142 to the power source 146. With additional reference to FIG. 4, in one embodiment, the body 138 of the implantable catheter 102 can further define one or more electrical conduit lumens 152A/B through which the one or more electrical conduits 150A/B can traverse. Accordingly, in some embodiments, the implantable catheter 102 can have a tri-lumen configuration, including the first lumen 136, through which medicament passes, and a second and third lumen 152A/B configured to house the piezoelectric element cables or wires 150A/B.

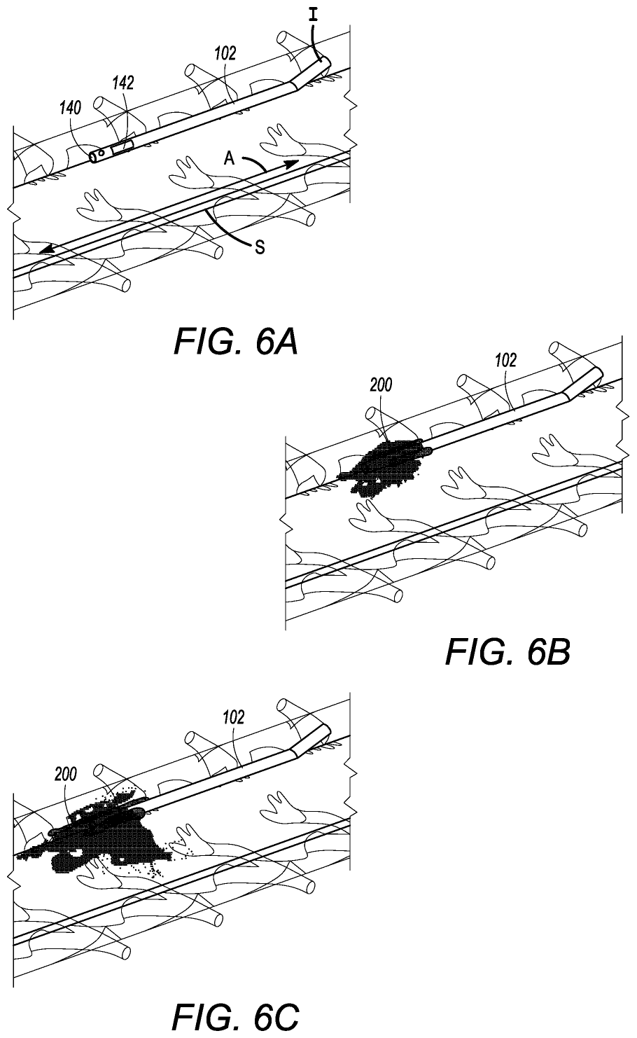

FIG. 6A depicts a catheter 102 having a piezoelectric element 142 configured to selectively oscillate during medicament administration positioned within a subarachnoid space of a patient. In particular, the catheter 102 enters the subarachnoid space at an insertion site I, and extends substantially parallel to a longitudinal axis A of the patient's spinal column S, thereby enabling intrathecal delivery of medicament through an infusion port 140 of the catheter 102. FIGS. 6B and 6C depict the catheter 102 as the medicament 200 exits the infusion port 140 and flows into the subarachnoid space. Specifically, FIG. 6B depicts the dispersion of medicament 200 after approximately 7.5 seconds of initiating infusion, and FIG. 6C depicts the dispersion of medicament 200 after approximately 15 seconds of initiating infusion.

The dispersion of medicament 200 delivered via catheter 102 into the subarachnoid space (and other areas within the human body) can be simulated using fluid dynamics modeling methods such as finite volume, finite element, or finite difference techniques for finding approximate solutions to systems of partial differential equations. In the case of intrathecal delivery, the system of partial differential equations that model conservation of mass and momentum, also known as Navier-Strokes equations, can simulate cerebrospinal fluid flow. To be more precise, the equations for laminar, oscillating flow of an incompressible fluid with properties similar to water at body temperature can be used to simulate medicament 200 delivery scenarios. Medicament 200 dispersion can further be modeled using various techniques including the Eulerian passive scaler approach or the Lagrangian particle approach.

FIGS. 5A-C and FIGS. 6A-C represent predictions of respective volumes of dispersed clouds of medicament 200 in an idealized intrathecal space geometry with cerebrospinal fluid that oscillates according to a sine function with a 1 Hz frequency and 3 mL/s amplitude. With a nominal catheter 100 (such as that depicted in FIGS. 5A-C), at a time of 30 seconds after the start of a bolus infusion at 1 mL/hr, the infuse medicament can occupy a volume of approximately 25 mm³. By contrast, for an embodiment where the catheter 102 includes a piezoelectric element 142 configured to oscillate during medicament administration to improve dispersion of the medicament 200 within the cerebrospinal fluid (such as that depicted in FIGS. 6A-C), with all other model parameters kept constant, the infused medicament 200 can occupy a volume of approximately 500 mm³. Thus, it can be seen that the piezoelectric element 142, as described above, can have the effect of increasing the volume of dispersed medicament 200 approximately twenty times that of a catheter without a piezoelectric element.

Accordingly, in some embodiments, the piezoelectric element 142 is configured to oscillate for a predefined period of time in relation to administration of the medicament 200. For example, in one embodiment, the piezoelectric element 142 is configured to oscillate during the entire time that medicament 200 is flowing through the lumen 136 and passing into the cerebrospinal fluid, and for a short time thereafter, thereby enabling dispersion of the medicament 200 beyond a slower moving flow of cerebrospinal fluid within a boundary layer immediately surrounding the implantable catheter 102. In some embodiments, the piezoelectric element 142 can be configured to oscillate for a period of between about 15 seconds and about 30 seconds; although other periods of time are also contemplated. For example, in some embodiments, bolus deliveries may be longer than a period of 30 seconds, and the presence of the medicament 200 in the cerebrospinal fluid can last for several hours after infusion. During this time, the piezoelectric element 142 can oscillate continuously, or cycle on and off to encourage mixing while preserving a battery life of the power source 146. The piezoelectric element 142 can be configured to oscillate within a frequency range of between about 20 kHz and about 60 kHz; for example, in one embodiment, the piezoelectric element 142 can be configured to oscillate at about 41 kHz.

In some embodiments, oscillation of the piezoelectric element 142 can be timed to correspond with the insertion of a needle into the implantable port 104, thereby inferring the administration of medicament. For example, in some embodiments, the implantable port 104 can include a needle detection sensor 154. In some embodiments, the needle detection sensor 154 can be a mechanical switch, acoustic sensor, optical or photoelectric sensor, ultrasonic sensor, pressure sensor, capacitive sensor, Hall effect sensor, to name just a few. The needle detection sensor 154 can send a signal to a processor 156 upon detection of a needle entering the first or second chamber 116/124. Thereafter, the processor 156 can activate the piezoelectric element 142 to encourage mixing of the medicament 200 with the cerebrospinal fluid. In some embodiments, the piezoelectric element 142 can be activated during the entire time that the needle is detected by the needle detection sensor 154, and optionally for a predetermined time after a detected removal of the needle by the needle detection sensor 154.

In some embodiments, oscillation of the piezoelectric element 142 can be timed to correspond with a detected flow of medicament 200. For example, in some embodiments, the implantable port 104 and/or catheter 102 can include a flow sensor 158 configured to detect a flow of medicament. In some embodiments, the flow sensor 158 can be a pressure sensor, a variable resistor, strain gauge, inductance coil, Hall effect sensor, resonant circuit, capacitive sensor, sonically based sensor, light based sensor, or a sensor configured to measure energy requirements of an associated pump, to name just a few. The flow sensor 158 can send a signal to the processor 156 upon detection of a flow of medicament. Thereafter, the processor 156 can activate the piezoelectric element 142 to encourage mixing of the medicament with the cerebrospinal fluid. In some embodiments, the piezoelectric element 142 can be activated during the entire time that the flow of medicament is detected by the flow sensor 158, and optionally for a predetermined time after the flow sensor 158 ceases to detect a flow of medicament.

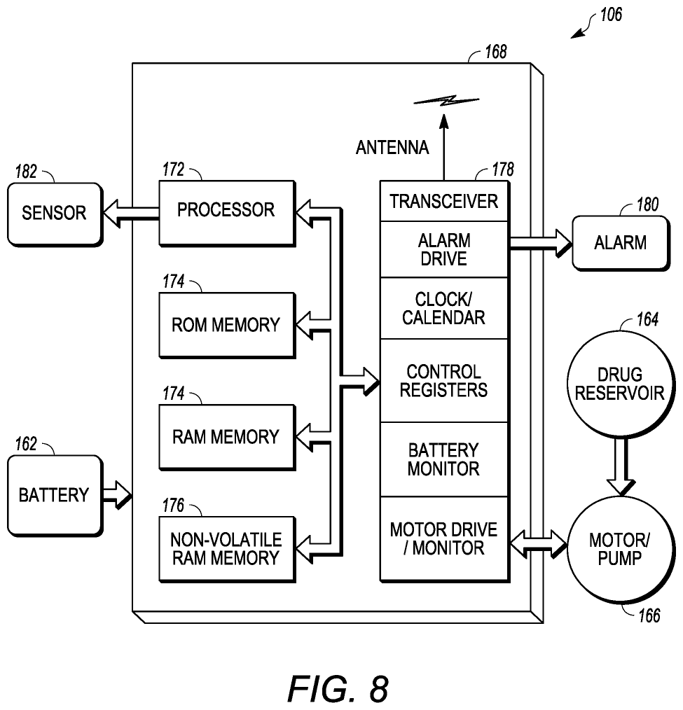

As an alternative to an implantable port 104, in some embodiments, the catheter 102 can be operably coupled to an implantable pump 106. Referring to FIG. 7, a medical device 100 including an implantable medical pump 106 is depicted in accordance with an embodiment of the disclosure. FIG. 7 depicts an exploded perspective view of the implantable medical pump 106. FIG. 8 is a block diagram of the implantable medical pump 106.

The implantable medical pump 106 can generally include a housing 160, power source 162, medicament reservoir 164, medicament pump 166, and electronics 168. The housing 160 can be constructed of a material that is biocompatible and hermetically sealed, such as titanium, tantalum, stainless steel, plastic, ceramic, or the like. The power source 162 can be a battery, such as a lithium ion battery. The power source 162 can be carried in the housing 160, and can be selected to operate the medicament pump 166 and other electronics 168, including a piezoelectric element 142 of the catheter 102.

The medicament reservoir 164 can be carried by the housing 160 and can be configured to contain medicament. In one embodiment, medicament within the medicament reservoir 164 can be accessed via an access port 170. Accordingly, the access port 170 can be utilized to refill, empty or exchange the fluid within the medicament reservoir 164.

The medicament pump 166 can be carried by the housing 160. The medicament pump 166 can be in fluid communication with the medicament reservoir 164 and can be in electrical communication with the electronics 168. The medicament pump 166 is a pump that is sufficient for infusing medicament to the patient, such as a piston pump, a peristaltic pump, a pump powered by a stepper motor, a pump powered by an AC motor, a pump powered by a DC motor, an electrostatic diaphragm, a piezo electric motor, a solenoid, a shape memory alloy, or the like.

The electronics 168 are carried in the housing, and can be in electrical communication with the power source 162, the medicament pump 166 and optionally the piezoelectric element 142 of the implantable catheter 102. In one embodiment, the electronics 168 can include a processor 172, a memory 174, 176, and transceiver circuitry 178. In one embodiment, the processor 172 can be an application-specific integrated circuit (ASIC) state machine, gate array, controller, or the like. The electronics 168 can be generally configured to control infusion of medicament according to programmed parameters or a specified treatment protocol. The programmed parameters are specified treatment protocol can be stored in the memory 174. The transceiver circuitry 178 can be configured to receive information from and transmit information to optional external sensors and an optional external programmer. In one embodiment, the electronics 168 can be further configured to operate a number of other features, such as a patient alarm 180.

In one embodiment, the electronics 168 can additionally be configured to include or communicate with one or more sensors 182 configured to serve as a triggering mechanism for activation and timing of the piezoelectric element 142. Examples of the one or more sensors 182 include a needle detection sensor 154, a flow detection sensor 158, or a physiological sensor which can be configured to communicate with the processor 172 to selectively activate the piezoelectric element 142 to encourage mixing of the medicament with the cerebrospinal fluid.

As an alternative to an implantable port 104 or implantable pump 106, in yet another embodiments, the catheter 102 can be a transdermal or transcutaneous catheter configured to be inserted through the patient's skin and into a subarachnoid, epidural, intrathecal, or intracranial space of the patient for delivery of medicament, such that the proximal end 128 of the catheter 102 is positioned exterior to the body of the patient, and the distal end 108, including the infusion port 140 and piezoelectric element 142 are positioned internal to the patient. Such a transcutaneous embodiment can be particularly adapted for temporary or single use applications.

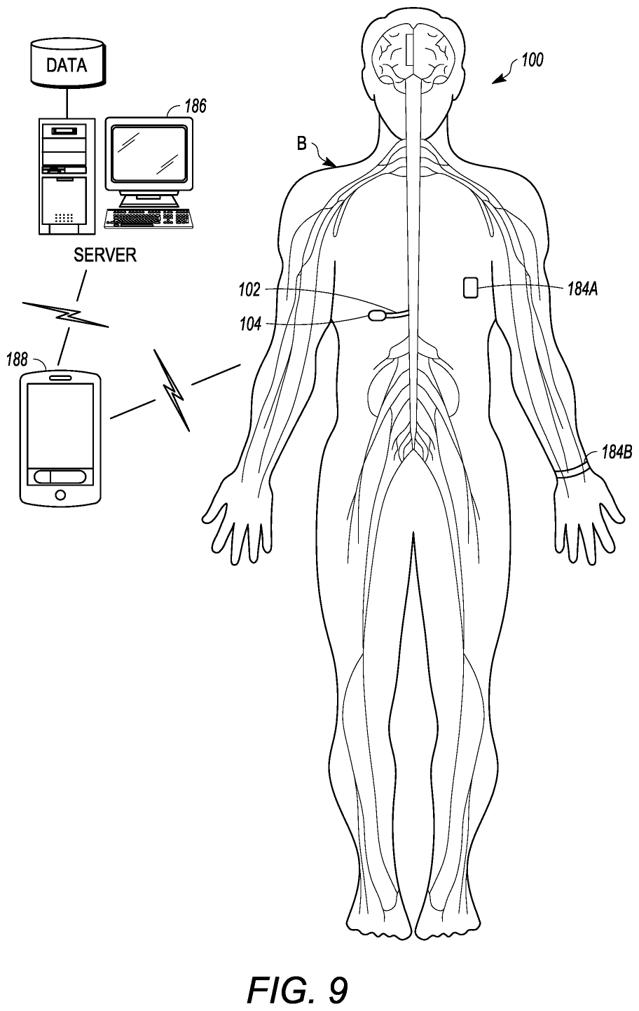

In some embodiments, the medical system 100 can include a number of components both internal and external to the patient. For example, as depicted in FIG. 9, the medical system 100 can include an implantable catheter 102, a port 104, one or more physiological sensors 184 (such as embedded sensor 184 as depicted in FIG. 2, or external sensors 184A and 184B as depicted in FIG. 9), and an optional server 186 and optional external programmer 188.

The physiological sensors 184 can be any sensor configured to monitor one or more physiological conditions affecting cerebrospinal fluid circulation. Examples of physiological sensors 184 include a heart rate monitor, pulse oximeter, respiratory sensor, perspiration sensor, posture orientation sensor, motion sensor, accelerometer, or the like. In some embodiments, an increase in patient activity (as measured by an increase in heart rate, respiratory rate, etc., can infer an increase in frequency of cerebrospinal fluid oscillations, which in turn can improve mixing of the medicaments with the cerebrospinal fluid.

In one embodiment, one or more physiological sensors 184 can be incorporated into the port 104 or pump 106. In one embodiment, a physiological sensor 184 can be worn by the patient (e.g., a smart watch, wristband tracker, sensors embedded in clothing, etc.), carried by the patient (e.g., a smart phone, mobile computing device, etc.), or positioned in proximity to the patient (e.g., a stationary monitor, etc.). In one embodiment, the external programmer 188 can include one or more physiological sensors 184. Data from the one or more physiological sensors 184 can be utilized to determine an increased rate of activity by the patient, which can infer an increase in the frequency of cerebrospinal fluid oscillations. In some embodiments, conditions sensed by the one or more sensors 184 can be communicated to the processor 156, 172, which can in turn send a signal to selectively activate the piezoelectric element 142.

In some embodiments, the physiological sensor 184 can be configured to monitor one or more conditions of the patient continuously. In other embodiments, the physiological sensor 184 is limited to sensing patient conditions during one or more periods of time in which a specified quantity of medicament is to be administered. Data collected by the one or more physiological sensors 184 can be utilized to establish patient specific baselines or thresholds; for example, a resting state baseline (e.g., less than 70 bpm) and an active state threshold (e.g., greater than 90 bpm). In one embodiment, the resting state baseline or active state threshold can be utilized as a trigger to activate the piezoelectric element 142. In one embodiment, the one or more baselines or thresholds can be utilized to activate the piezoelectric element 142; for example, different baselines or thresholds can be established during different times of the day. In one embodiment, activation of the piezoelectric element 142 can be triggered based on a rate of change in the activity (e.g., using a derivative of a sensed or measured physiological condition of the patient). In one embodiment, the one or more establish baselines or threshold can serve as an initial default, and can be manually adjusted by a clinician or patient via the external programmer 188. For example, in one embodiment, a patient can input activity schedule information (e.g., workout times, etc.) and adjust the baseline or thresholds accordingly.

FIGS. 2, 3 and 7 depict a medical device 100 in which the piezoelectric element 142 is positioned proximately to an infusion port 140. Other configurations and placements of the piezoelectric element 142 relative to the infusion port 140 are also contemplated. For example, the piezoelectric element 142 can be positioned distal to the infusion port 140, on the distal tip 108 of the catheter 102, or adjacent to or surrounding the infusion port 140. In some embodiments, multiple piezoelectric elements 142 can be utilized along a section of catheter 102.

In general, the piezoelectric element 142 can include elementary cells configured to change dimension when an electrical potential (e.g., power source 146, 162) is applied. In some embodiments, the piezoelectric element 142 can include a stack of thin piezoceramic layers configured to extend when a voltage is applied. In some embodiments, the piezoelectric element 142 can be incorporated into a bimorph or other form of bending plate 190.

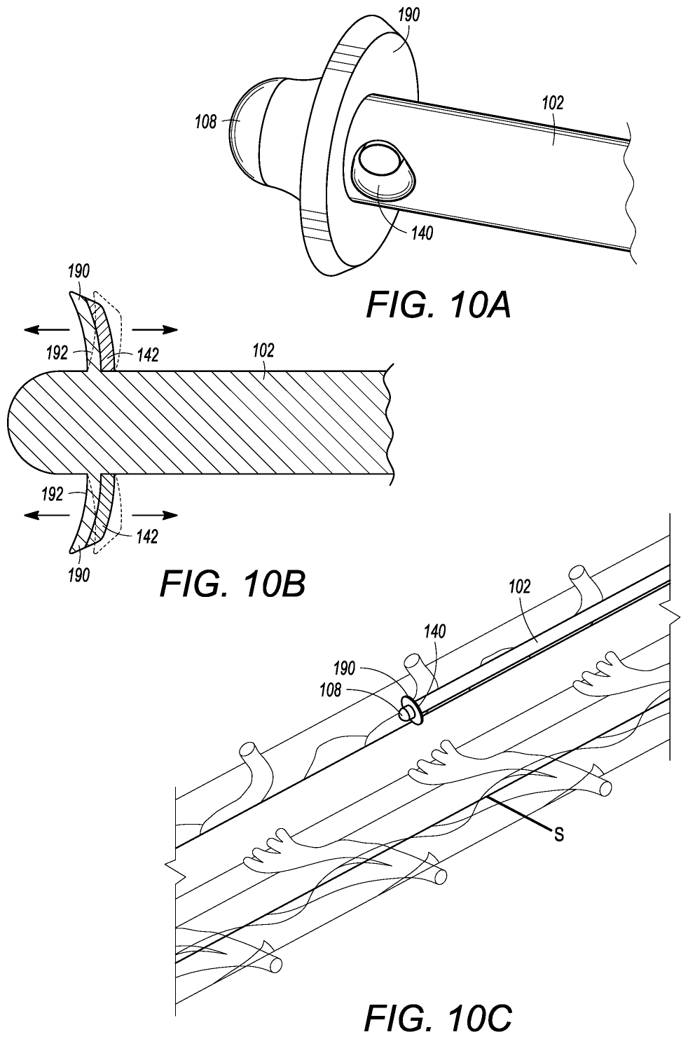

FIGS. 10A-C depict an example embodiment of a catheter 102 including a bending plate 190. As depicted, the bending plate 190 can include a first piezoelectric element layer 142 in contact with a second layer 192 of a different material. Application of an electrical potential to the piezoelectric element layer 142 can cause the bending plate 190 to rapidly oscillate. From a cross-sectional view (as depicted in FIG. 10B), the bending plate 190 can be seen as bending laterally adjacent to the infusion port 140. As depicted in FIG. 10C, the utilization of a bending plate 190 incorporating a piezoelectric element layer 142 can further enhance a rapid dispersion of the medicament within the cerebrospinal fluid during medicament administration.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An medical device configured to improve medicament dispersion, the medical device comprising:
an implantable catheter having a distal end configured to be positioned within a flow of cerebrospinal fluid of a patient, a proximal end, and a body defining a lumen extending lengthwise along the implantable catheter configured to enable a flow of medicament from the proximal end to an infusion port positioned in proximity to the distal end, the implantable catheter further including a piezoelectric element positioned in proximity to the infusion port configured to selectively oscillate during medicament administration to improve dispersion of the medicament within the cerebrospinal fluid.

2. The medical device of claim 1, wherein the piezoelectric element is configured to oscillate for a predefined period of time in relation to administration of the medicament.

3. The medical device of claim 1, wherein the medical device further includes one or more physiological sensors configured to monitor one or more physiological conditions of the patient to time oscillation of the piezoelectric element to correspond with an inference of heightened cerebrospinal fluid oscillations.

4. The medical device of claim 1, wherein the one or more physiological sensors is configured to monitor at least one of a heart rate or respiratory rate of the patient.

5. The medical device of claim 1, wherein the proximal end of the implantable catheter is operably coupled to an implantable port configured to subcutaneously receive the medicament.

6. The medical device of claim 5, wherein medical device further comprises a needle detection sensor configured to detect an insertion of a needle into the implantable port to time oscillation of the piezoelectric element.

7. The medical device of claim 1, wherein medical device further comprises a medicament flow sensor configured to detect a flow of medicament to time oscillation of the piezoelectric element.

8. The medical device of claim 1, further comprising an implantable power source configured to power the piezoelectric element.

9. The medical device of claim 8, wherein the implantable power source is configured to be inductively charged through a skin of the patient.

10. The medical device of claim 8, wherein the body of the implantable catheter further defines one or more electrical conduit lumen extending lengthwise along the implantable catheter configured to house the one or more electrical conduit electrically coupling the implantable power source to the piezoelectric element.

11. The medical device of claim 1, wherein the piezoelectric element includes a bending plate configured to impart fluid motion in the cerebrospinal fluid and medicament surrounding the implantable catheter during administration of the medicament.

12. An medical device configured to improve medicament dispersion within a flow of cerebrospinal fluid of the patient, the medical device comprising:
an implantable catheter having a distal end configured to be positioned within the flow of cerebrospinal fluid, a proximal end, a body defining a lumen configured to enable a flow of medicament to an infusion port positioned in proximity to the distal end, and a piezoelectric element positioned in proximity to the infusion port; and
an implantable port in fluid communication with the implantable catheter, configured to receive medicament from a medicament source;
wherein the piezoelectric element includes an oscillating surface configured to impart fluid motion in the cerebrospinal fluid and medicament surrounding the implantable catheter to encourage dispersion of the medicament beyond a slower moving flow of cerebrospinal fluid within a boundary layer immediately surrounding the implantable catheter.

13. The medical device of claim 12, wherein the piezoelectric element is configured to oscillate for a predefined period of time in relation to administration of the medicament.

14. The medical device of claim 12, wherein the medical device further includes one or more physiological sensors configured to monitor one or more physiological conditions of the patient to time oscillation of the piezoelectric element to correspond with an inference of heightened cerebrospinal fluid oscillations.

15. The medical device of claim 12, wherein the one or more physiological sensors is configured to monitor at least one of a heart rate or respiratory rate of the patient.

16. The medical device of claim 15, wherein the implantable port includes a needle detection sensor configured to detect an insertion of a needle into the implantable port to time oscillation of the piezoelectric element.

17. The medical device of claim 12, wherein the implantable port includes a medicament flow sensor configured to detect a flow of medicament to time oscillation of the piezoelectric element.

18. The medical device of claim 12, wherein the implantable port includes a power source configured to power the piezoelectric element.

19. The medical device of claim 18, wherein the implantable power source is configured to be inductively charged through a skin of the patient.

20. A method of improving medicament dispersion comprising:
administering medicament into a flow of cerebrospinal fluid of a patient via an implantable catheter having a distal end configured to be positioned within the flow of cerebrospinal fluid, a proximal end, a body defining a lumen configured to enable a flow of medicament to an infusion port positioned in proximity to the distal end, and a piezoelectric element positioned in proximity to the infusion port; and
selectively oscillating a surface of the piezo electric element to impart fluid motion in the cerebrospinal fluid and medicament surrounding the implantable catheter to encourage dispersion of the medicament beyond a slower moving flow of cerebrospinal fluid within a boundary layer immediately surrounding the implantable catheter.
